(19) United States
(12) Patent Application Publication
(10) Pub. No.: US 2026/0198790 A1
(43) Pub. Date: Jul. 16, 2026

(54) MICROCAVITY-ARRAYED BLOOD PRESSURE DETECTION SYSTEM BASED ON A DIGITAL OPTICAL FREQUENCY DUAL-COMB

(71) Applicant: SUN YAT-SEN UNIVERSITY, Guangdong (CN)

(72) Inventors: Zhaohui LI, Guangdong (CN); Ruifeng ZHONG, Guangdong (CN); Jingshun PAN, Guangdong (CN)

(73) Assignee: SUN YAT-SEN UNIVERSITY, Guangdong (CN)

(21) Appl. No.: 19/558,464

(22) Filed: Mar. 6, 2026

Related U.S. Application Data
(63) Continuation-in-part of application No. PCT/CN2024/117328, filed on Sep. 6, 2024.

(30) Foreign Application Priority Data
Sep. 8, 2023 (CN) 202311153520.1

(51) Int. Cl. A61B 5/021; A61B 5/00

(52) U.S. Cl. CPC A61B 5/02116; A61B 5/0082

(57) ABSTRACT

A microcavity-arrayed blood pressure detection system based on digital optical frequency dual-comb is provided. An input end of an optical intensity modulator is connected to a laser and a waveform generator. An output end of the optical intensity modulator, a polarization controller, and an on-chip optical microcavity array are connected sequentially. The optical intensity modulator modulates single-frequency light based on an electrical modulation signal to obtain an optical frequency dual-comb signal which enters the on-chip optical microcavity array through the polarization controller. The on-chip optical microcavity array is fitted onto an artery position to be detected and includes sensing units, each sensing unit generates a separate pulse signal, the on-chip optical microcavity array obtains a sensing signal based on the optical frequency dual-comb signal and pulsatile compression at the artery position to be detected, and a signal processing device obtains blood pressure value information based on the sensing signal.

### CROSS-REFERENCE TO RELATED APPLICATION

This application is a continuation-in-part of international PCT application serial no. PCT/CN2024/117328, filed on Sep. 6, 2024, which claims the priority benefit of China application no. 202311153520.1, filed on Sep. 8, 2023. The entirety of each of the above mentioned patent applications is hereby incorporated by reference herein and made a part of this specification.

### BACKGROUND

### Technical Field

This application relates to the field of optical sensing technologies, and more specifically, to an microcavity-arrayed blood pressure detection system based on a digital optical frequency dual-comb.

### Description of Related Art

Blood pressure is an important parameter index for assessing human health, as hypertension can cause a series of diseases such as coronary heart disease, angina pectoris, myocardial infarction, and diabetes. In the treatment of hypertension, continuous blood pressure monitoring data plays a significant guiding role.

In the prior art, continuous blood pressure measurement can only be implemented by invasive measurement methods based on catheterization. Such methods measure blood pressure at an arterial site by using a pressure transducer. Other common methods, such as a Korotkoff sound method and an oscillometric method, perform blood pressure measurement in a discrete manner with relatively long time intervals between measurements, and require an inflatable cuff to apply an external force to assist the measurement. In contrast, a pulse wave velocity method for blood pressure measurement requires two measurement points to be arranged on a body surface, and implements noninvasive and continuous blood pressure estimation by measuring a time delay of a pulse wave between the two measurement points, thereby replacing an inflatable cuff used in conventional blood pressure measurement. Generally, in most pulse wave velocity methods, two measurement points are mainly used at the heart and the wrist. Long-distance measurement imposes higher requirements on positional stability and spatial dimensions.

A development trend of optical sensing requires a demodulation system to have real-time detection, high multiplexing capacity, high precision, and the like. This, in turn, imposes higher requirements on a laser source used in a sensing system, such as a high sweep-frequency rate, a wide scanning range, and a narrow instantaneous linewidth. Therefore, the development of sensing technology is currently limited not only by devices themselves but also, to a great extent, by the light source. An optical frequency comb is an extremely promising new type of light source. However, conventional optical frequency comb teeth are generated using mode-locked lasers, which have significant deficiencies in terms of accuracy and speed, and lack flexible adjustability. In addition, conventional dual-combs require two lasers, which greatly increases the complexity of the system.

### SUMMARY

Embodiments of this application are intended to provide an microcavity-arrayed blood pressure detection system and method based on a digital optical frequency dual-comb, which can achieve the technical effect of improving the accuracy and convenience of blood pressure measurement.

In a first aspect, an embodiment of this application provides an microcavity-arrayed blood pressure detection system based on a digital optical frequency dual-comb, comprising a laser, a waveform generator, an optical intensity modulator, a polarization controller, an on-chip optical microcavity array, and a signal processing device, where
an input end of the optical intensity modulator is respectively connected to the laser and the waveform generator, an output end of the optical intensity modulator, the polarization controller, and the on-chip optical microcavity array are connected in sequence, the laser emits single-frequency light of a preset frequency, the waveform generator generates an electrical modulation signal based on a preset time-domain signal, and the preset time-domain signal is obtained by inverse Fourier transform of and superposition of two sets of frequency comb tooth signals with preset comb tooth spacings and a preset frequency difference;
the optical intensity modulator modulates the single-frequency light based on the electrical modulation signal to obtain an optical frequency dual-comb signal; and the optical frequency dual-comb signal enters the on-chip optical microcavity array through the polarization controller, the on-chip optical microcavity array is fitted onto an artery position to be detected and comprises multiple sensing units, each of the multiple sensing units generates a separate pulse signal, the on-chip optical microcavity array obtains a sensing signal based on the optical frequency dual-comb signal and pulsatile compression at the artery position to be detected, and the signal processing device obtains blood pressure value information based on the sensing signal.

In the above implementation, this microcavity-arrayed blood pressure detection system based on a digital optical frequency dual-comb obtains an optical frequency dual-comb signal through the laser, the a waveform generator, and the optical intensity modulator, fits the on-chip optical microcavity array onto an artery position to be detected, inputs the optical frequency dual-comb signal into the on-chip optical microcavity array, and acquires a sensing signal under the pulsatile compression at the artery position to be detected, and obtains blood pressure value information based on the sensing signal. The microcavity-arrayed blood pressure detection system based on a digital optical frequency dual-comb uses the digital optical frequency dual-comb as a light source, offering advantages of a high bandwidth, high precision, and high speed which are freely adjustable. In addition, the microcavity-arrayed blood pressure detection system based on a digital optical frequency dual-comb can measure a time delay between two points over an extremely short distance by employing an arrayed optical microring, thereby enhancing system stability on one hand and saving spatial dimensions for greater convenience on the other hand. As a result, the microcavity-arrayed blood pressure detection system based on a digital optical frequency dual-comb can achieve the technical effect of improving the accuracy and convenience of blood pressure measurement.

Further, the two sets of frequency comb tooth signals with the preset comb tooth spacings and the preset frequency difference are obtained as follows: generating two pulse square wave signals by the waveform generator, where the two pulse square wave signals implement phase synchronization based on a same reference clock and have a fixed difference in a repeated frequency period; and subsequently, inputting the two pulse square wave signals into a Mach-Zehnder electro-optic modulator to periodically and alternately modulate an input continuous narrow linewidth laser light, and using a spectral slicing property of the two pulse square wave signals to form the two sets of frequency comb tooth signals with different comb tooth spacings and a comb tooth spacing difference consistent to a repeated frequency period difference of the square waves in a frequency domain.

Further, the two sets of frequency comb tooth signals with the preset comb tooth spacings and the preset frequency difference are obtained as follows: a pseudo-random sequence code is generated by the waveform generator, and then the pseudo-random sequence code is used to generate two sets of frequency comb teeth with different comb tooth spacings and the preset frequency difference.

Further, the signal processing device comprises an erbium-doped optical fiber amplifier, and the erbium-doped optical fiber amplifier is connected to the on-chip optical microcavity array.

In the above implementation, due to inherent losses in the on-chip optical microcavity array and additional losses from packaging and coupling, the sensing signal (specifically the optical sensing signal passed through the chip) is amplified by the erbium-doped optical fiber amplifier.

Further, the signal processing device comprises a coherent receiver, and the coherent receiver is respectively connected to the erbium-doped optical fiber amplifier and the laser.

Further, the signal processing device comprises an oscilloscope, and the oscilloscope is connected to the coherent receiver.

In the above implementation, an amplified sensing signal enters the coherent receiver. When the laser emits the single-frequency light, split reference light is sent to the coherent receiver. The sensing signal and the reference light enter the coherent receiver for demodulation together, and finally, frequency comb data is acquired through the oscilloscope to reconstruct a pulse wave waveform.

Further, the sensing signal satisfies the following relationship:

$$\frac{Δλ}{λ} = \frac{Δ l}{l} = \frac{Δ n}{n} ;$$

where λ is a resonant wavelength of a microring resonator in the on-chip optical microcavity array, Δλ is a variation in the resonant wavelength, Δl is a deformation of a waveguide, l is an original total length of the waveguide, Δn is a variation in a refractive index of the waveguide, and n is an original refractive index of the waveguide.

Further, the on-chip optical microcavity array comprises two sensing units, each sensing unit generates a set of sensing signals, and blood pressure value information is obtained by analyzing two sets of sensing signals and a continuous time difference between the two sets of sensing signals based on a pulse wave velocity method.

Further, the artery position to be detected is a radial artery position.

In a second aspect, an embodiment of this application provides an microcavity-arrayed blood pressure detection method based on a digital optical frequency dual-comb, which is used in the microcavity-arrayed blood pressure detection system based on a digital optical frequency dual-comb described in any of the first aspect. The detection method comprises:
emitting single-frequency light of a preset frequency through a laser;
generating an electrical modulation signal through a waveform generator based on a preset time-domain signal, where the preset time-domain signal is obtained by performing inverse Fourier transform and superposition on two sets of frequency comb tooth signals with preset comb tooth spacings and a preset frequency difference;
modulating the single-frequency light based on the electrical modulation signal to obtain an optical frequency dual-comb signal;
transmitting the optical frequency dual-comb signal to an on-chip optical microcavity array to obtain a sensing signal, where the on-chip optical microcavity array is fitted onto an artery position to be detected; and
obtaining blood pressure value information at the artery position to be detected based on the sensing signal.

Further, before the step of generating the electrical modulation signal through the waveform generator based on the preset time-domain signal, the method further comprises:
generating two pulse square wave signals through a waveform generator, where the two pulse square wave signals are phase-synchronized based on a same reference clock and have a fixed difference in repeated frequency period;
inputting the two pulse square wave signals into a Mach-Zehnder electro-optic modulator to periodically and alternately modulate an input continuous narrow linewidth laser light, and using a spectral slicing property of the pulse square wave signals to form two sets of frequency comb tooth signals with different comb tooth spacings and a comb tooth spacing difference consistent to a repeated frequency period difference of the square waves in a frequency domain; and
converting the two sets of frequency comb teeth into two sets of time-domain signals through fast inverse Fourier transform and superposing the two sets of time-domain signals, to obtain a preset time-domain signal.

Further, before the step of generating the electrical modulation signal through the waveform generator based on the preset time-domain signal, the method further comprises:
generating two sets of frequency comb teeth with different comb tooth spacings and a preset frequency difference using a pseudo-random sequence code; and
converting the two sets of frequency comb teeth into two sets of time-domain signals through fast inverse Fourier transform and superposing the two sets of time-domain signals, to obtain a preset time-domain signal.

Further, after the step of obtaining the blood pressure value information at the artery position to be detected based on the sensing signal, the method further comprises:

Obtaining, based on the sensing signal, pulse wave waveform information by monitoring frequency shifts using a frequency comb.

Other features and advantages disclosed in this application will be described in the following specification, or some features and advantages may be inferred from or clearly determined by the specification, or may be learned by implementing the above-disclosed technology of this application.

To make the above objectives, features, and advantages of this application more obvious and understandable, preferred embodiments are enumerated below and described in detail with reference to accompanying drawings.

### BRIEF DESCRIPTION OF THE DRAWINGS

To more clearly illustrate technical solutions of the embodiments of this application, accompanying drawings to be used in the embodiments of this application will be briefly described below. It should be understood that the following drawings only show some embodiments of this application and should not be considered as limiting the scope. For those skilled in the art, other related drawings can also be obtained based on these drawings without creative work.

FIG. 1 is a schematic structural diagram of an microcavity-arrayed blood pressure detection system based on a digital optical frequency dual-comb according to an embodiment of this application.

FIG. 2 is a schematic structural diagram of an on-chip optical microcavity array according to an embodiment of this application.

FIG. 3 is a schematic flow diagram of an microcavity-arrayed blood pressure detection method based on a digital optical frequency dual-comb according to an embodiment of this application.

FIG. 4 is a schematic flow diagram of another microcavity-arrayed blood pressure detection method based on a digital optical frequency dual-comb according to an embodiment of this application.

FIG. 5 is a schematic flow diagram of another microcavity-arrayed blood pressure detection method based on a digital optical frequency dual-comb according to an embodiment of this application.

FIG. 6 is a schematic diagram of two sets of frequency comb teeth according to an embodiment of this application.

FIG. 7 is a schematic diagram of a frequency comb demodulation principle according to an embodiment of this application.

FIG. 8 is a schematic diagram of long-term stable measurement data according to an embodiment of this application.

FIG. 9 is a schematic diagram of a time difference between two sets of pulse waves according to an embodiment of this application.

FIG. 10 is a schematic diagram of detection of blood pressure value information versus time according to an embodiment of this application.

### REFERENCE NUMERALS

laser 100; waveform generator 200; optical intensity modulator 300; polarization controller 400; on-chip optical microcavity array 500; sensing unit 510; optical fiber 520; signal processing device 600; erbium-doped optical fiber amplifier 610; coherent receiver 620; oscilloscope 630.

### DESCRIPTION OF THE EMBODIMENTS

The following clearly and completely describes technical solutions in embodiments of this application with reference to accompanying drawings in the embodiments of this application. Apparently, the described embodiments are only some rather than all of the embodiments of this application. Generally, components of embodiments of this application described and shown in the accompanying drawings herein may be arranged and designed in various configurations. Therefore, the following detailed descriptions of the embodiments of this application provided in the accompanying drawings are not intended to limit the scope of the claimed application, but merely to represent selected embodiments of this application. All other embodiments obtained by those skilled in the art based on the embodiments of this application without creative efforts shall fall within the protection scope of this application.

In this application, orientations or positional relationships indicated by terms “upper”, “lower”, “left”, “right”, “front”, “rear”, “top”, “bottom”, “inner”, “outer”, “vertical”, “horizontal”, “transverse”, “longitudinal”, and the like are based on orientations or positional relationships shown in the drawings. These terms are mainly intended to better describe this application and embodiments therein, and are not intended to limit that an apparatus, an element, or a component that is indicated must have a specific orientation, or be constructed and operated in a specific orientation.

In addition, some of the above terms may be used to indicate other meanings in addition to the orientation or positional relationship. For example, the term “upper” may also be used to indicate a dependency or connection relationship in some cases. Those skilled in the art may understand specific meanings of these terms in this application according to specific cases.

In addition, terms “mount”, “dispose”, “provided with”, “connect”, and “connected” should be construed broadly. For example, a connection may be a firm connection, a detachable connection, or an integral construction; a mechanical connection or a point connection; a direct connection, an indirect connection through an intermediary medium, or an internal communication between two apparatuses, elements, or components. For those skilled in the art, the specific meanings of the above terms in this application can be understood according to particular circumstances.

In addition, terms “first”, “second”, and the like are mainly used to distinguish between different apparatuses, elements, or components (which may or may not be the same in type and construction) and are not intended to indicate or imply relative importance and a quantity of the apparatuses, elements, or components indicated. In addition, unless otherwise specified, “multiple” means two or more.

### Embodiment 1

This embodiment of this application is based on an microcavity-arrayed blood pressure detection system based on a digital optical frequency dual-comb, which can be used in continuous blood pressure monitoring. This microcavity-arrayed blood pressure detection system based on the digital optical frequency dual-comb obtains an optical frequency dual-comb signal through a laser, a waveform generator, and an optical intensity modulator, fits an on-chip optical microcavity array onto an artery position to be detected, inputs the optical frequency dual-comb signal into the on-chip optical microcavity array, and acquires a sensing signal under the pulsatile compression at the artery position to be detected, and obtains blood pressure value information based on the sensing signal. The microcavity-arrayed blood pressure detection system based on the digital optical frequency dual-comb uses the digital optical frequency dual-comb as a light source, offering advantages of a high bandwidth, high precision, and high speed which are freely adjustable. In addition, the microcavity-arrayed blood pressure detection system based on the digital optical frequency dual-comb can measure a time delay between two points over an extremely short distance by employing an arrayed optical microring, thereby enhancing system stability on one hand and saving spatial dimensions for greater convenience on the other hand. As a result, the microcavity-arrayed blood pressure detection system based on the digital optical frequency dual-comb can achieve the technical effect of improving the accuracy and convenience of blood pressure measurement.

In recent years, with the rapid development of an on-chip optical process, a light source technology, and optical information processing, it has become possible to implement higher-quality sensing and detection in biomedicine and other fields using more sensitive and faster optical methods. The high integration and ease of arraying of an optical microcavity, combined with a highly flexible digital optical frequency dual comb, enable more precise and easier blood pressure measurement compared to various conventional blood pressure detection methods.

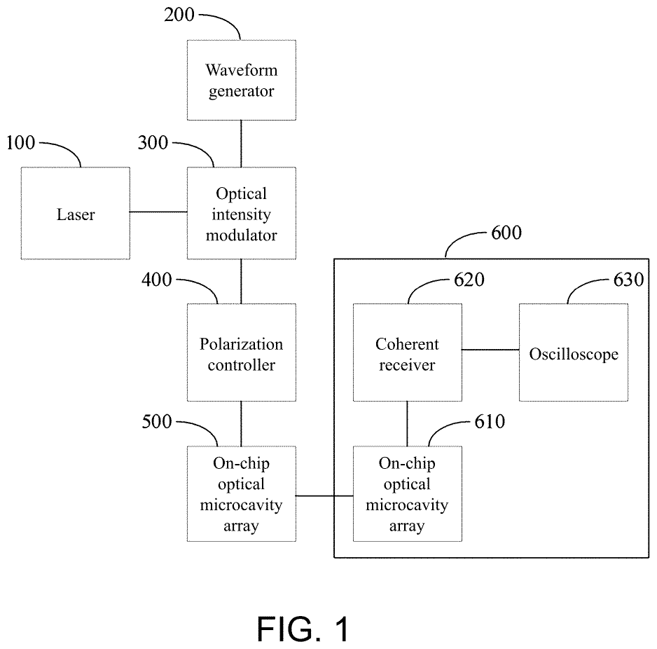

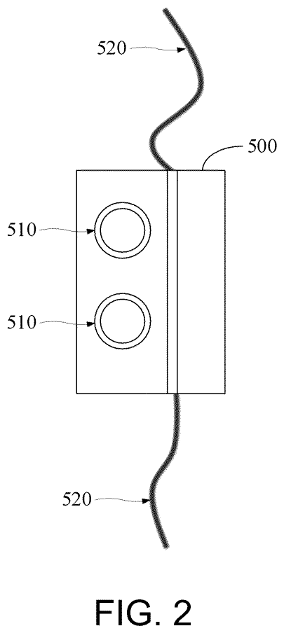

Referring to FIG. 1 and FIG. 2, FIG. 1 is a schematic structural diagram of an microcavity-arrayed blood pressure detection system based on a digital optical frequency dual-comb according to an embodiment of this application, and FIG. 2 is a schematic structural diagram of an on-chip optical microcavity array according to an embodiment of this application. The microcavity-arrayed blood pressure detection system based on a digital optical frequency dual-comb comprises a laser 100, a waveform generator 200, an optical intensity modulator 300, a polarization controller 400, an on-chip optical microcavity array 500, and a signal processing device 600.

For example, an input end of the optical intensity modulator 300 is respectively connected to the laser 100 and the waveform generator 200. An output end of the optical intensity modulator 300, the polarization controller 400, and the on-chip optical microcavity array 500 are connected sequentially. The laser 100 emits single-frequency light of a preset frequency, and the waveform generator 200 generates an electrical modulation signal based on a preset time-domain signal. The preset time-domain signal is obtained by performing inverse Fourier transform and superposition on two sets of frequency comb tooth signals with preset comb tooth spacings and a preset frequency difference.

For example, the optical intensity modulator 300 modulates the single-frequency light based on the electrical modulation signal to obtain an optical frequency dual-comb signal. After passing through the polarization controller 400, the optical frequency dual-comb signal enters the on-chip optical microcavity array 500, and the on-chip optical microcavity array 500 is fitted onto an artery position to be detected. The on-chip optical microcavity array 500 comprises multiple sensing units, each generating a separate pulse signal. The on-chip optical microcavity array 500 acquires a sensing signal based on the optical frequency dual-comb signal and pulsatile compression at the artery position to be detected, and the signal processing device 600 obtains blood pressure value information based on the sensing signal.

For example, the signal processing device 600 comprises an erbium-doped optical fiber amplifier 610, and the erbium-doped optical fiber amplifier 610 is connected to the on-chip optical microcavity array 500.

For example, due to inherent losses in the on-chip optical microcavity array 500 and additional losses from packaging and coupling, the sensing signal (specifically the optical sensing signal passed through the chip) is amplified by the erbium-doped optical fiber amplifier.

For example, the signal processing device 600 further comprises a coherent receiver 620, and the coherent receiver 620 is respectively connected to the erbium-doped optical fiber amplifier 610 and the laser 100.

For example, the signal processing device 600 further comprises an oscilloscope 630, and the oscilloscope is connected to the coherent receiver 620.

For example, an amplified sensing signal enters the coherent receiver 620. When the laser 100 emits the single-frequency light, split reference light is sent to the coherent receiver 620. The sensing signal and the reference light enter the coherent receiver 620 for demodulation together, and finally, frequency comb data is acquired through the oscilloscope 630 to reconstruct a pulse wave waveform.

For example, the sensing signal satisfies the following relationship:

$$\frac{Δλ}{λ} = \frac{Δ l}{l} = \frac{Δ n}{n} ;$$

where λ is a resonant wavelength of a microring resonator in the on-chip optical microcavity array, Δλ is a variation in the resonant wavelength, Δl is a deformation of a waveguide, l is an original total length of the waveguide, Δn is a variation in a refractive index of the waveguide, and n is an original refractive index of the waveguide.

For example, the on-chip optical microcavity array 500 comprises two sensing units 510, and each sensing unit 510 generates a set of sensing signals. Blood pressure value information is obtained by analyzing two sets of sensing signals and a continuous time difference between the two sets of sensing signals based on a pulse wave velocity method. Optionally, both ends of the on-chip optical microcavity array 500 are connected to corresponding optical fibers 520 respectively.

For example, the artery position to be detected is a radial artery position.

### Embodiment 2

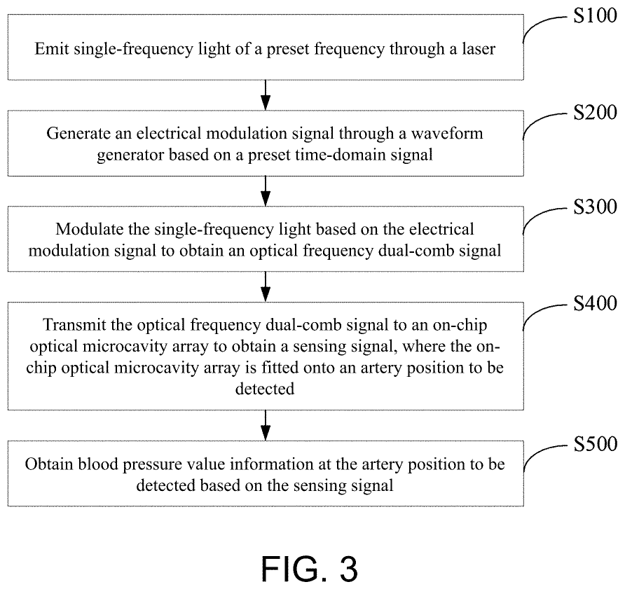

Refer to FIG. 3. FIG. 3 is a schematic flow diagram of an microcavity-arrayed blood pressure detection method based on a digital optical frequency dual-comb according to an embodiment of this application, which is used in the microcavity-arrayed blood pressure detection system based on a digital optical frequency dual-comb as shown in FIG. 1 and FIG. 2. The detection method comprises the following steps:
S100: Emitting single-frequency light of a preset frequency through a laser;
S200: Generating an electrical modulation signal through a waveform generator based on a preset time-domain signal, where the preset time-domain signal is obtained by performing inverse Fourier transform and superposition on two sets of frequency comb tooth signals with preset comb tooth spacings and a preset frequency difference;
S300: Modulating the single-frequency light based on the electrical modulation signal to obtain an optical frequency dual-comb signal;
S400: Transmitting the optical frequency dual-comb signal to an on-chip optical microcavity array to obtain a sensing signal, where the on-chip optical microcavity array is fitted onto an artery position to be detected; and
S500: Obtaining blood pressure value information at the artery position to be detected based on the sensing signal.

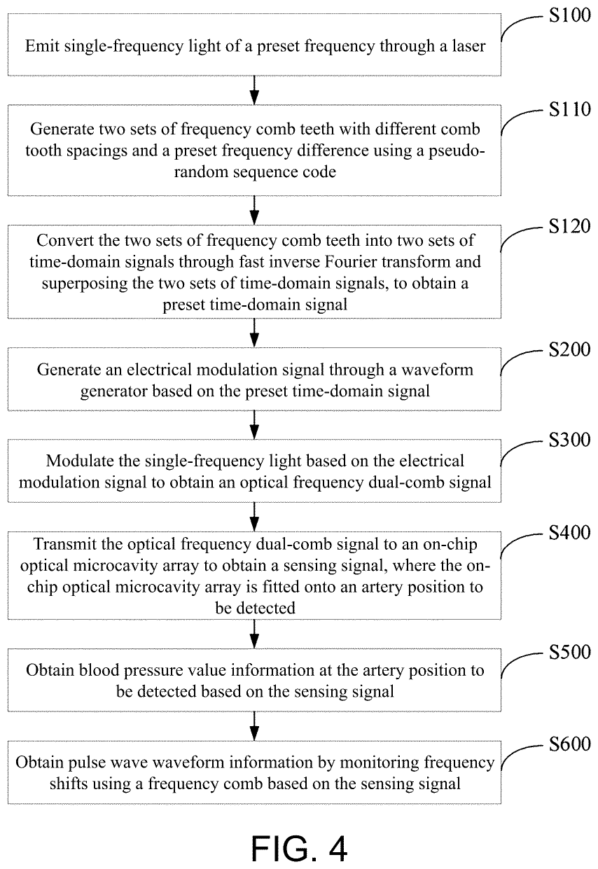

Refer to FIG. 4. FIG. 4 is a schematic flow diagram of another microcavity-arrayed blood pressure detection method based on a digital optical frequency dual-comb according to an embodiment of this application.

For example, before the steps S200: generating an electrical modulation signal through a waveform generator based on a preset time-domain signal, the method further comprises:
S110: Generating two sets of frequency comb teeth with different comb tooth spacings and a preset frequency difference using a pseudo-random sequence code; and
S120: Converting the two sets of frequency comb teeth into two sets of time-domain signals through fast inverse Fourier transform and superposing the two sets of time-domain signals, to obtain a preset time-domain signal.

For example, after the steps S500: obtaining blood pressure value information at the artery position to be detected based on the sensing signal, the method further comprises:
S600: obtaining, based on the sensing signal, pulse wave waveform information by monitoring frequency shifts using a frequency comb.

In some implementations, the microcavity-arrayed blood pressure detection system based on a digital optical frequency dual-comb provided in this embodiment of this application can perform parallel measurement on pulse waves at two positions of a radial artery using the on-chip optical microcavity array. Because a conventional single-wavelength laser cannot meet requirements for arrayed detection, a digital optical frequency dual-comb with a high bandwidth, high precision, and high speed which are freely adjustable is introduced for detection and demodulation. The pulse wave signal is acquired, a time delay between two different nearby positions is extracted, and combined with a mathematical model relating a time difference to a blood pressure, so that more accurate systolic and diastolic pressure values can be obtained. Therefore, the microcavity-arrayed blood pressure detection system based on the digital optical frequency dual-comb improves the discontinuity of current blood pressure measurement and the instability and inconvenience of a conventional pulse wave velocity method.

In some implementations, with reference to FIG. 1 to FIG. 4, the microcavity-arrayed blood pressure detection method based on the digital optical frequency dual-comb provided in this application operates as follows:

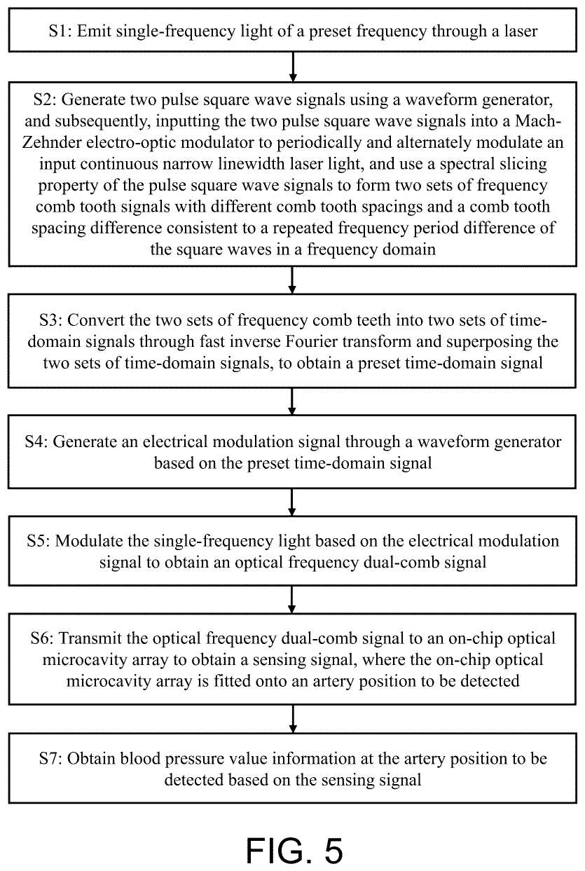

The single-frequency light with a frequency f0 is emitted by the laser and enters the optical intensity modulator for modulation; and
two sets of frequency comb teeth with a bandwidth of 2 GHz and comb tooth spacings of 3.920 MHz and 3.913 MHz, respectively (with a frequency difference of approximately 7 kHz), are generated using the pseudo-random sequence code by a computer terminal. FIG. 5 is a schematic diagram of two sets of frequency comb teeth according to an embodiment of this application.

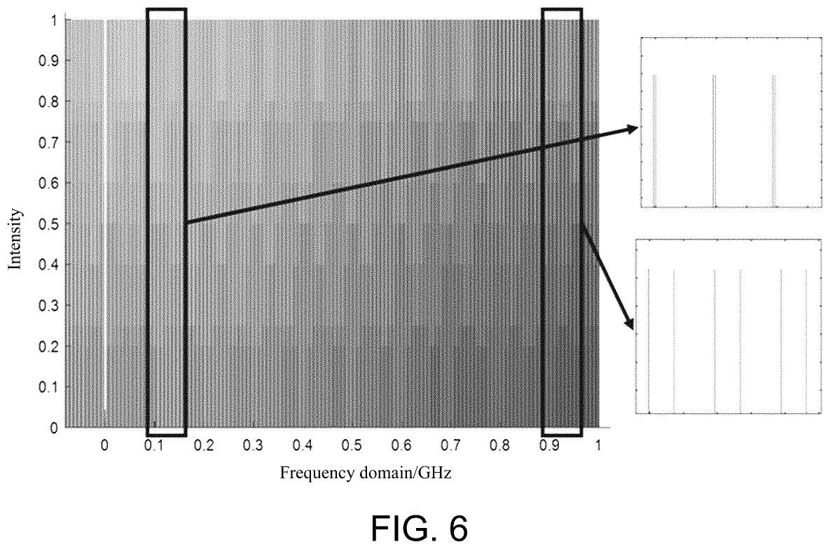

For example, FIG. 6 shows a schematic diagram illustrating an increasing frequency difference between two sets of comb teeth in a simulated frequency domain.

The two sets of frequency comb teeth (frequency domain signals) are converted into time-domain signals through the fast inverse Fourier transform and superposed to generate the preset time-domain signal. A code for the generated preset time-domain signal is then imported into a 60 GS/s signal generator to produce an electric signal consistent with the code. This electric signal is used as a modulation signal for the optical intensity modulator to modulate signal light split from the laser.

The optical frequency dual-comb signal (optical signal) after passing through the optical intensity modulator is a series of optical frequency comb teeth in a frequency domain consistent with that of the previous design. The dual-comb mutually beats at a demodulation end, implementing a bandwidth requirement for down-conversion reduction at a demodulation end, increasing a sampling rate, and improving time resolution of pulse wave detection.

After passing through the polarization controller, the optical frequency dual-comb signal directly enters the on-chip optical microcavity array. Each sensing unit in the array can generate a separate pulse signal. The on-chip optical microcavity array is fitted onto the radial artery along a trend of the radial artery, and the pulsation of the radial artery compresses the on-chip optical microcavity array, causing deformation of the microcavity of the on-chip optical microcavity array, which leads to a change in a refractive index and a drift in a resonance peak. According to a microring resonance formula 2πnR=mλ, the following relationship can be derived:

$$\frac{Δλ}{λ} = \frac{Δ l}{l} = \frac{Δ n}{n} ;$$

where λ is a resonant wavelength of a microring resonator, Δλ is a variation in a resonant wavelength, Δl is a deformation of a waveguide, l is an original total length of the waveguide, Δn is a variation in a refractive index of the waveguide, and n is an original refractive index of the waveguide.

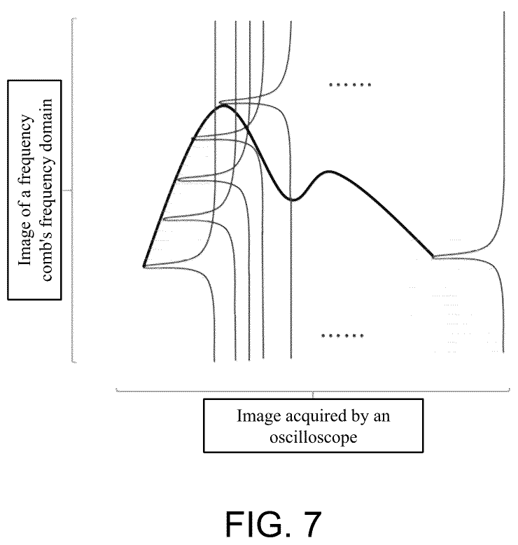

A pulse wave waveform is reconstructed by monitoring frequency shifts using a frequency comb. FIG. 7 is a schematic diagram of a frequency comb demodulation principle according to an embodiment of this application.

For example, as shown in FIG. 7, a vertical axis represents a frequency-domain image obtained after single-frame dual-comb beating, from which amplitude information of a pulse wave at a given moment is extracted according to the resonant wavelength in the frequency domain, where an amplitude intensity corresponds to a resonance peak shift, and a comb tooth spacing defines vertical resolution of the pulse wave. A horizontal axis represents continuous pulse information obtained under continuous long-term sampling, and a sampling rate of each spectral image frame defines the lateral resolution of the pulse wave. A bold solid line represents a reconstructed pulse wave signal.

Because sensing units of the on-chip optical microcavity array (taking two sensing units as an example) are placed in time order, and each sensing unit generates a set of sensing signals, there is a corresponding time difference between the two sets of sensing signals. Based on a principle of the pulse wave velocity method, a continuous time difference is analyzed and substituted into a wave velocity-blood pressure model formula to obtain the blood pressure value.

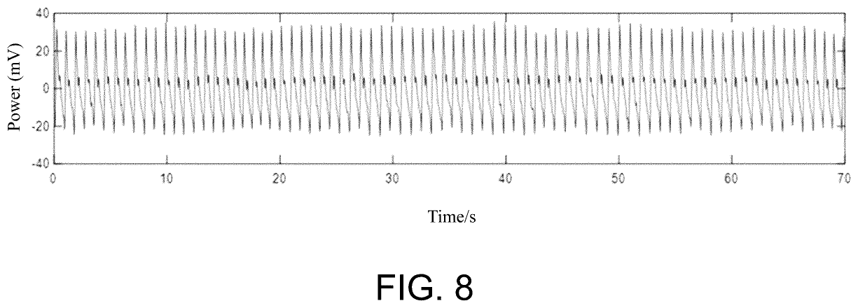

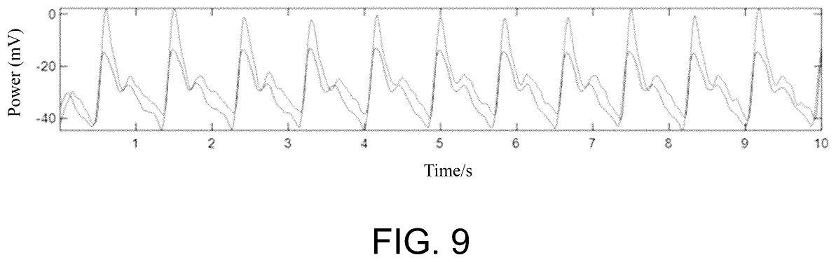

Due to inherent losses in a chip and additional losses from packaging and coupling, subsequent amplification is required through the erbium-doped optical fiber amplifier. An amplified optical signal and previously split reference light enter the coherent receiver for demodulation together, and finally, frequency comb data is acquired through the oscilloscope to reconstruct the waveform. A principle is shown in FIG. 8 and FIG. 9. FIG. 8 is a schematic diagram of long-term stable measurement data according to an embodiment of this application. FIG. 9 is a schematic diagram of a time difference between two sets of pulse waves according to an embodiment of this application.

For example, a pulse wave velocity method is based on a Moens-Korteweg equation, which describes a relationship between a pulse wave velocity (PWV) and an elastic modulus Eᵢₙ of an arterial wall, as expressed below:

$$PWV = \sqrt{{E}_{in} h / 2 ρ r} ;$$

where h is an arterial wall thickness, ρ is a blood density, and r is an arterial radius. The elastic modulus Eᵢₙ of the arterial wall is directly related to blood pressure, with a relational expression as follows:

$${E}_{in} = {E}_{0} {e}^{{γ}^{P}} \text{?}$$

$$\text{?} \text{indicates text missing or illegible when filed}$$

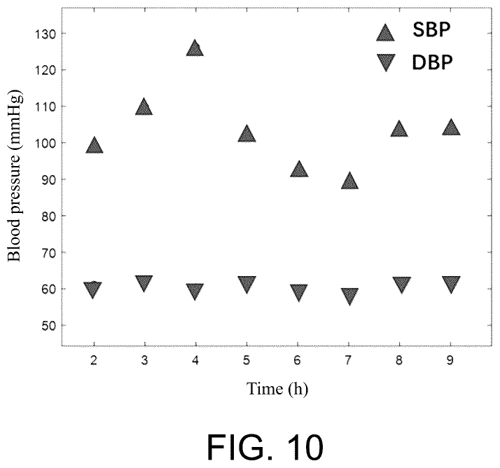

In combination with a Bramwell-Hill formula, values of systolic blood pressure (SBP) and diastolic blood pressure (DBP) can be further obtained:

$$DBP = \frac{1}{3} {SBP}_{0} + \frac{2}{3} {DBP}_{0} + Aln ( \frac{{PTT}_{0}}{PTT} ) - \frac{{SBP}_{0} - {DBP}_{0}}{3} {( \frac{{PTT}_{0}}{PTT} )}^{2} \text{?}$$

$$SBP = DBP + ( {SBP}_{0} - {DBP}_{0} ) {( \frac{{PTT}_{0}}{PTT} )}^{2} \text{?}$$

$$\text{?} \text{indicates text missing or illegible when filed}$$

[100] where PTT is pulse transit time, A is an empirically determined mean value for individual-specific differences obtained via large-scale data analysis, SBP₀, DBP₀, and PTT₀ are preset initial values for the systolic blood pressure, diastolic blood pressure, and the pulse transit time, respectively, and may be calibrated together with other initial values. Accordingly, as long as the time difference between each pair of points is identified using the frequency comb method, continuous non-invasive blood pressure measurement can be implemented. FIG. 10 shows an average blood pressure value within 10 seconds every hour.

In some implementation scenarios, actual experiments have shown that the on-chip optical microcavity combined with the digital optical frequency dual-comb system in this application can implement precise pulse detection, accurately identify characteristic parameter points of pulse waves such as a main wave, a dicrotic wave, and dicrotic notch, as well as a time delay between two sets of data. The use of the digital optical frequency comb system makes arrayed pulse detection simpler. In addition, through the deduction of the above formula, accurate systolic and diastolic blood pressure values can be obtained, which is a more accurate and simpler optical arrayed blood pressure measurement method.

### Embodiment 3

This embodiment provides an microcavity-arrayed blood pressure detection method based on a digital optical frequency dual-comb. Other content of this embodiment is similar to Embodiment 2, with a difference being a manner for generating two sets of frequency comb tooth signals. In this embodiment, a pseudo-random binary sequence (PRBS) signal relied upon in Embodiment 2 is abandoned. Instead, two pulse square wave signals with precisely controllable parameters are designed and generated using a field-programmable gate array (FPGA) in combination with a high-precision reference clock source, a high-speed pulse shaping module, and a power amplification module. The two pulse square wave signals implement phase synchronization based on a same reference clock, with only a fixed difference in a repeated frequency period. This repeated frequency period can be flexibly set within 1 KHz to 50 KHz, or adjusted to any other preset value according to actual application scenarios (such as spectral resolution, a detection bandwidth, or sensing accuracy requirements). In addition, a half-peak width of the square wave is strictly controlled within a range of 0.01 ns to 1 ns. A pulse edge characteristic is optimized through the pulse shaping module, ensuring rise time and fall time are ≤0.001 ns, thus preventing pulse broadening that affects the purity of frequency domain comb teeth. Subsequently, these two parameter-matched pulse square wave signals are respectively input to corresponding drive ports of a Mach-Zehnder electro-optic modulator (MZM) to periodically and alternately modulate an input continuous narrow linewidth laser light. A spectral slicing property of the square wave signals is used to form two sets of frequency comb tooth signals with different comb tooth spacings and a comb tooth spacing difference consistent to a repeated frequency period difference of the square waves in the frequency domain, thereby implementing effective generation of the optical frequency dual-comb. This design directly simplifies a PRBS signal generation process, eliminating complex steps of PRBS signal encoding, sequence length optimization, error checking, and synchronization calibration in the original solution, and retaining only basic steps of signal frequency division, shaping, and amplification.

Specifically, in this embodiment, as shown in FIG. 3 and FIG. 5, the microcavity-arrayed blood pressure detection method based on the digital optical frequency dual-comb comprise the following steps:
Step S1: emitting single-frequency light of a preset frequency through a laser;
Step S2: generating two pulse square wave signals using a waveform generator, where the two pulse square wave signals implement phase synchronization based on a same reference clock and have a fixed difference in a repeated frequency period; and subsequently, inputting the two pulse square wave signals into a Mach-Zehnder electro-optic modulator to periodically and alternately modulate an input continuous narrow linewidth laser light, and using a spectral slicing property of the pulse square wave signals to form the two sets of frequency comb tooth signals with different comb tooth spacings and a comb tooth spacing difference consistent to a repeated frequency period difference of the square waves in a frequency domain;
Step S3: converting the two sets of frequency comb teeth into two sets of time-domain signals through fast inverse Fourier transform and superposing the two sets of time-domain signals, to obtain a preset time-domain signal;
Step S4: generating an electrical modulation signal through a waveform generator based on the preset time-domain signal;
Step S5: modulating single-frequency light based on the electrical modulation signal to obtain an optical frequency dual-comb signal;
Step S6: transmitting the optical frequency dual-comb signal to an on-chip optical microcavity array to obtain a sensing signal, with the on-chip optical microcavity array fitted onto an artery to be detected; and
Step S7: obtaining blood pressure value information at the artery position to be detected based on the sensing signal.

In this embodiment, on the one hand, a newly added pulse square wave path greatly simplifies logic for generating a modulation signal, significantly reducing device volume, power consumption, and hardware costs, and high-performance FPGA and dedicated verification chips are not required, which is more suitable for portable devices and low-cost research scenarios; on the other hand, parameter tuning flexibility and system stability are significantly improved, a comb tooth spacing difference and a half-peak width can be separately controlled, and a phase and amplitude stability of the pulse square wave, with the pulse square wave as a deterministic signal, are superior to a PRBS signal, a signal-to-noise ratio of a generated optical frequency dual-comb is improved by 3-5 dB, and the comb uniformity is better, providing a high-quality signal foundation for subsequent detection and analysis. In addition, this solution is compatible with a peripheral of the original solution and has strong scalability.

In all embodiments of this application, “large” and “small” are relative, “more” and “less” are relative, and “upper” and “lower” are relative. The descriptions of such relative terms are not further elaborated in the embodiments of this application.

It should be understood that the terms “in this embodiment,” “in the embodiment of this application,” or “in an optional embodiment” throughout the specification mean that specific features, structures, or characteristics related to the embodiment are included in at least one embodiment of this application. Therefore, phrases “in this embodiment,” “in the embodiment of this application,” or “in an optional embodiment” appearing in various places throughout the specification do not necessarily mean the same embodiment. Further, these specific features, structures, or characteristics can be combined in any suitable way in one or more embodiments. Those skilled in the art should appreciate that all the embodiments described in this specification are optional embodiments, and the related actions and modules are not necessarily required by this application.

It should be understood that sequence numbers of the foregoing processes do not mean execution sequences in various embodiments of this application. The execution sequences of the processes should be determined according to functions and internal logic of the processes, and should not be construed as any limitation on the implementation processes of the embodiments of this application.

The foregoing descriptions are merely specific implementations of this application, but are not intended to limit the protection scope of this application. Any variation or replacement readily figured out by those skilled in the art within the technical scope disclosed in this application shall fall within the protection scope of this application. Therefore, the protection scope of this application shall be subject to the protection scope of the claims.

What is claimed is:

1. An microcavity-arrayed blood pressure detection system based on a digital optical frequency dual-comb, comprising: a laser, a waveform generator, an optical intensity modulator, a polarization controller, an on-chip optical microcavity array, and a signal processing device,
wherein an input end of the optical intensity modulator is respectively connected to the laser and the waveform generator, an output end of the optical intensity modulator, the polarization controller, and the on-chip optical microcavity array are connected in sequence, the laser emits single-frequency light of a preset frequency, the waveform generator generates an electrical modulation signal based on a preset time-domain signal, and the preset time-domain signal is obtained by inverse Fourier transform of and superposition of two sets of frequency comb tooth signals with preset comb tooth spacings and a preset frequency difference; the optical intensity modulator modulates the single-frequency light based on the electrical modulation signal to obtain an optical frequency dual-comb signal; and the optical frequency dual-comb signal enters the on-chip optical microcavity array after passing through the polarization controller, the on-chip optical microcavity array is fitted onto an artery position to be detected and comprises multiple sensing units, each of the multiple sensing units generates a separate pulse signal, the on-chip optical microcavity array obtains a sensing signal based on the optical frequency dual-comb signal and pulsatile compression at the artery position to be detected, and the signal processing device obtains blood pressure value information based on the sensing signal.

2. The microcavity-arrayed blood pressure detection system based on the digital optical frequency dual-comb according to claim 1, wherein the two sets of frequency comb tooth signals with the preset comb tooth spacings and the preset frequency difference are obtained as follows:
generating two pulse square wave signals by the waveform generator, wherein the two pulse square wave signals implement phase synchronization based on a same reference clock and have a fixed difference in a repeated frequency period; and
subsequently, inputting the two pulse square wave signals into a Mach-Zehnder electro-optic modulator to periodically and alternately modulate an input continuous narrow linewidth laser light, and using a spectral slicing property of the two pulse square wave signals to form the two sets of frequency comb tooth signals with different comb tooth spacings and a comb tooth spacing difference consistent to a repeated frequency period difference of square waves in a frequency domain.

3. The microcavity-arrayed blood pressure detection system based on the digital optical frequency dual-comb according to claim 2, wherein the signal processing device comprises an erbium-doped optical fiber amplifier, and the erbium-doped optical fiber amplifier is connected to the on-chip optical microcavity array.

4. The microcavity-arrayed blood pressure detection system based on the digital optical frequency dual-comb according to claim 3, wherein the signal processing device further comprises a coherent receiver, and the coherent receiver is respectively connected to the erbium-doped optical fiber amplifier and the laser.

5. The microcavity-arrayed blood pressure detection system based on the digital optical frequency dual-comb according to claim 4, wherein the signal processing device further comprises an oscilloscope, and the oscilloscope is connected to the coherent receiver.

6. The microcavity-arrayed blood pressure detection system based on the digital optical frequency dual-comb according to claim 2, wherein the sensing signal satisfies a following relationship:
$$\frac{Δλ}{λ} = \frac{Δ l}{l} = \frac{Δ n}{n} ;$$
wherein λ is a resonant wavelength of a microring resonator in the on-chip optical microcavity array, Δλ is a variation in the resonant wavelength, Δl is a deformation of a waveguide, l is an original total length of the waveguide, Δn is a variation in a refractive index of the waveguide, and n is an original refractive index of the waveguide.

7. The microcavity-arrayed blood pressure detection system based on the digital optical frequency dual-comb according to claim 2, wherein the on-chip optical microcavity array comprises two sensing units, each of the two sensing units generates a set of sensing signals, and blood pressure value information is obtained by analyzing two sets of sensing signals and a continuous time difference between the two sets of sensing signals based on a pulse wave velocity method.

8. The microcavity-arrayed blood pressure detection system based on the digital optical frequency dual-comb according to claim 2, wherein the artery position to be detected is a radial artery position.

9. The microcavity-arrayed blood pressure detection system based on the digital optical frequency dual-comb according to claim 1, wherein the two sets of frequency comb tooth signals with the preset comb tooth spacings and the preset frequency difference are obtained as follows:
generating a pseudo-random sequence code by the waveform generator, and then using the pseudo-random sequence code to generate two sets of frequency comb teeth with different comb tooth spacings and the preset frequency difference.

10. The microcavity-arrayed blood pressure detection system based on the digital optical frequency dual-comb according to claim 9, wherein the signal processing device comprises an erbium-doped optical fiber amplifier, and the erbium-doped optical fiber amplifier is connected to the on-chip optical microcavity array.

11. The microcavity-arrayed blood pressure detection system based on the digital optical frequency dual-comb according to claim 10, wherein the signal processing device further comprises a coherent receiver, and the coherent receiver is respectively connected to the erbium-doped optical fiber amplifier and the laser.

12. The microcavity-arrayed blood pressure detection system based on the digital optical frequency dual-comb according to claim 11, wherein the signal processing device further comprises an oscilloscope, and the oscilloscope is connected to the coherent receiver.

13. The microcavity-arrayed blood pressure detection system based on the digital optical frequency dual-comb according to claim 9, wherein the sensing signal satisfies a following relationship:
$$\frac{Δλ}{λ} = \frac{Δ l}{l} = \frac{Δ n}{n} ;$$
wherein λ is a resonant wavelength of a microring resonator in the on-chip optical microcavity array, Δλ is a variation in the resonant wavelength, Δl is a deformation of a waveguide, l is an original total length of the waveguide, Δn is a variation in a refractive index of the waveguide, and n is an original refractive index of the waveguide.

14. The microcavity-arrayed blood pressure detection system based on the digital optical frequency dual-comb according to claim 9, wherein the on-chip optical microcavity array comprises two sensing units, each of the two sensing units generates a set of sensing signals, and blood pressure value information is obtained by analyzing two sets of sensing signals and a continuous time difference between the two sets of sensing signals based on a pulse wave velocity method.

15. The microcavity-arrayed blood pressure detection system based on the digital optical frequency dual-comb according to claim 9, wherein the artery position to be detected is a radial artery position.
